(19) United States
(12) Patent Application Publication
(10) Pub. No.: US 2024/0078287 A1
(43) Pub. Date: Mar. 7, 2024

(54) Method To Minimize The Cost of Entraining A Target Limit Cycle

(71) Applicant: Arcascope Inc., Arlington, VA (US)

(72) Inventors: Olivia Walch, Falls Church, VA (US); Eric Canton, Eau Claire, WI (US); Kevin Hannay, Comfort, TX (US)

(21) Appl. No.: 17/614,985

(22) PCT Filed: May 28, 2021
(86) PCT No.: PCT/US2021/035024

§ 371 Date: Nov. 29, 2021

(51) Int. Cl. G06F 17/17; A61B 5/00

(52) U.S. Cl. CPC G06F 17/17; A61B 5/4857

(57) ABSTRACT

A goal of this invention is to minimize the cost of shifting a circadian state or entraining a state to a target cycle by identifying a preferred zeitgeber stimulus. Another goal of this invention is to provide a method to determine a person's circadian state trajectory given a stimulus time series.

### STATEMENT REGARDING FEDERALLY SPONSORED RESEARCH OR DEVELOPMENT

This invention was made with government support under award number 2R44CA236557-02A1 awarded by the National Cancer Institute. The government has certain rights in the invention.

### CROSS-REFERENCES TO RELATED APPLICATIONS

Not Applicable

### INCORPORATION-BY-REFERENCE OF MATERIAL SUBMITTED ON A COMPACT DISC

Not Applicable

### BACKGROUND

Circadian rhythms are endogenous rhythms with a periodicity of approximately 24 hours. This endogenous rhythm regulates human sleep and wake cycles, hormonal activities, body temperature, and hunger and digestion. However, external causes may prevent a person from operating with his or her preferred circadian rhythm. These external causes include shift work, travel to a new time zone, staying up late on the weekend, or any deviation from a fixed daily schedule. Ideally, a person's circadian rhythm will quickly correct in response to external factors; i.e., by returning to a baseline after an acute disturbance, such as a late night, or by adjusting (entraining) to a new time zone. However, the larger the time change, the more difficult it will be for an individual to entrain to a new circadian rhythm. Moreover, if the person is on a very irregular schedule, such as shift work, he or she may never entrain, and instead will be in a state of perpetual misalignment from his or her preferred rhythm.

An individual may receive a prescription on how to entrain to a new circadian rhythm, or control a rhythm that is not entrained to achieve some other effect, such as maximizing sleep duration or minimizing the number of hours where one is fatigued on a work shift. Such a prescription considers zeitgebers or “time-givers”. Zeitgebers are inputs, such as light, darkness, exercise, amongst others, that influence an individual's circadian clock. These inputs can affect both the central circadian clock, the suprachiasmatic nucleus (SCN), as well as peripheral circadian oscillators, such as clocks in the stomach, liver, etc. Light is the most important input to the central clock, but exercise and hormones, like melatonin, also have an effect. Food is a zeitgeber for the clocks in peripheral organs. Caffeine has also been reported to be a zeitgeber, particularly in the evening.

### BRIEF DESCRIPTION OF INVENTION

A goal of this invention is to minimize the cost of shifting a circadian state or entraining a state to a target cycle by identifying a preferred zeitgeber stimulus. Another goal of this invention is to provide a method to determine a person's circadian state trajectory given a stimulus time series.

### DESCRIPTION OF THE SEVERAL VIEWS OF THE DRAWINGS

The following detailed description will be better understood when read in conjunction with the appended drawings, in which there is shown example embodiments for the purposes of illustration. It should be understood, however, that the present disclosure is not limited to the precise arrangements and instrumentalities shown. In the drawings:

FIG. 1A is a graphical representation of a circadian rhythm and a zeitgeber;

FIG. 1B is a graphical representation of a circadian rhythm;

FIG. 2 shows an embodiment for a method to minimize cost to approximately entrain to a target circadian state;

FIG. 3A shows an embodiment of a method to simulate x(t);

FIG. 3B shows an embodiment of a method to simulate x(t);

FIG. 3C shows an embodiment of a method to simulate x(t);

FIG. 3D shows an embodiment of a method to simulate x(t);

FIG. 4 shows an embodiment of cost;

FIG. 5 shows an embodiment of a method to determine K(t);

FIG. 6 shows an embodiment of a method to determine K(t).

### DETAILED DESCRIPTION OF THE INVENTION

In the following detailed description, reference is made to the accompanying drawings, which form a part hereof. In the drawings, the use of similar or the same symbol in different drawings typically indicates similar or identical items, unless context dictates otherwise.

The illustrative embodiments described in the detailed description, drawings, and claims are not meant to be limiting. Other embodiments may be utilized, and other changes may be made, without departing from the spirit or scope of the subject matter presented here.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken as limiting.

The present application uses formal outline headings for clarity of presentation. However, it is to be understood that the outline headings are for presentation purposes, and that different types of subject matter may be discussed throughout the application (e.g., device(s)/structure(s) may be described under process(es)/operations heading(s) and/or process(es)/operations may be discussed under structure(s)/process(es) headings; and/or descriptions of single topics may span two or more topic headings). Hence, the use of the formal outline headings is not intended to be in any way limiting.

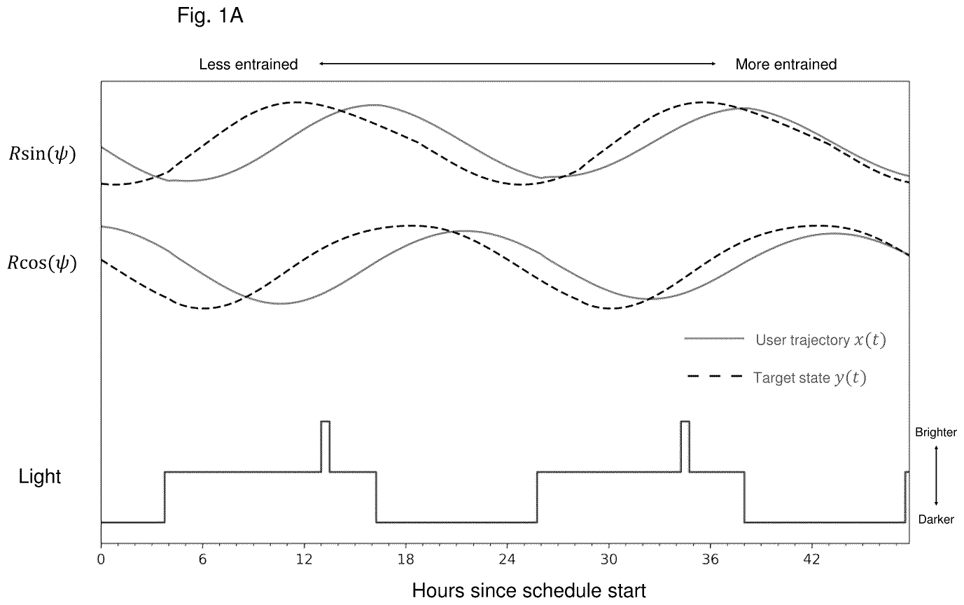

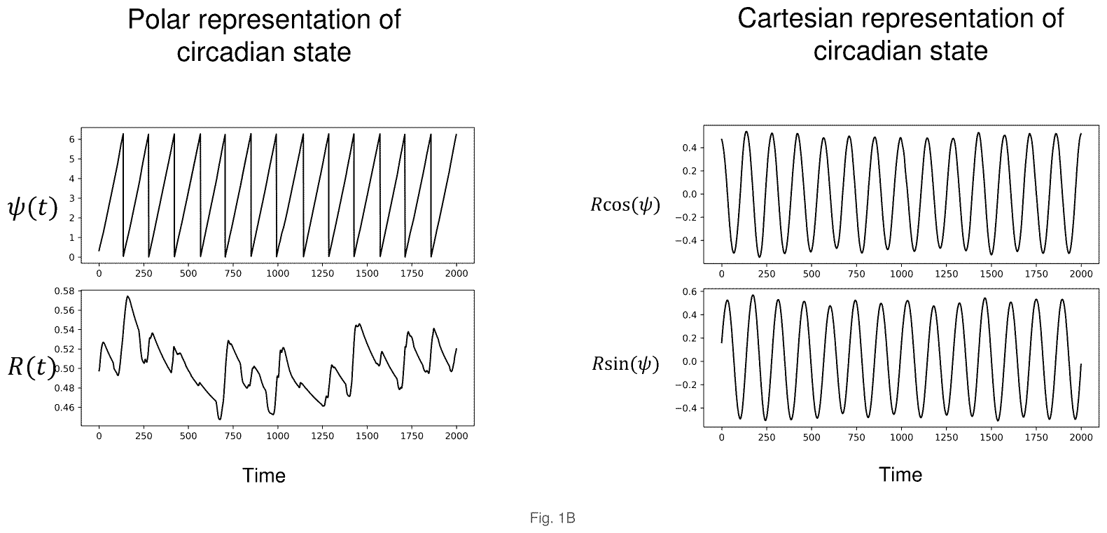

The invention provided for herein is a method to minimize a cost to approximately entrain to a target circadian state (100). Referring to FIG. 1A, a circadian rhythm can be represented by a curve that repeats with a period of approximately 24 hours. Here, the solid line represents a trajectory starting from an initial circadian state and the dashed line represents the target circadian state trajectory. The difference between the trajectory starting from the initial circadian state and the target circadian state trajectory, either at a point or over an entire interval, is the core component of the cost.

At least one zeitgeber is prescribed to shift or entrain the starting circadian state to a target circadian state. A zeitgeber may also be represented by a curve. Here, the stepped curves represent a or a series of zeitgebers.

To reduce potential confusion, the following glossary provides nomenclature for several frequently used terms within these specifications and claims with a view toward aiding in the comprehension of the invention described herein:
x(t₀)=x₀ (10); where x₀ is an initial circadian state at time t₀;
y(t) (30) is a target limit cycle state at time t;
x(t) (20) is a simulated circadian state at time t; x(t) may also be referred to as the circadian state trajectory;
K(t) (60) is the stimulus time series, or zeitgeber, applied to generate x(t) as a function of time, which can be either a stimulus history of inputs and outputs that can be used to track a historical circadian state, or a stimulus prescription designed to shift the clock prospectively in the future;
C (40) is a cost, which is a function of x(t), y(t), and K(t) and may be written as C(x(t), y(t), K(t)) (41), where cost can capture the difference between x(t) and y(t), as well as other factors;
K₀ (50) is an initial zeitgeber.

Referring to FIGS. 1A and 1B, state, as used above and throughout this specification and claims, is a vector; where “phase” and “amplitude” can be computed from components of the vector. For example, if the amplitude of x(t₀) is 0.9 while the phase is pi, the vector can be represented as x(t₀)=[0.9, pi]. State may include component variables other than amplitude and phase such as “light responsiveness”. For example, if “light responsiveness” at to is 0.2, the vector can be represented as x(t₀)=[0.9, pi, 0.2]. Generally, a state curve is represented as either amplitude×cos(phase) or amplitude×sin(phase); equivalently R×cos(psi) and R×sin(psi), where R is amplitude and psi is phase.

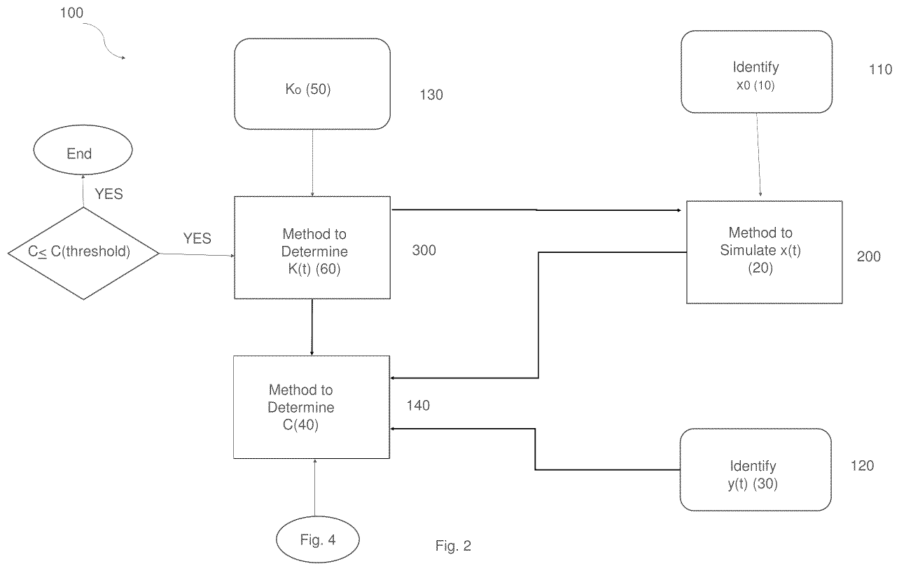

Referring to FIG. 2, an embodiment for a method to minimize cost to approximately entrain to a target circadian state (100) is comprised of: identifying a x₀(110); identifying a y(t) (120); prescribing an initial zeitgeber, K₀ (50) either at time t or over a time range [tᵢ, tⱼ] (130); a method to determine a zeitgeber, K(t) (60), either at time t or over a time range [tᵢ, tⱼ] (300); a method to simulate x(t) (20) in response to a zeitgeber K(t) (60), either at time t or over a time range [tᵢ, tⱼ] (200); a method to determine C (40) over the time range [tᵢ, tⱼ] (140). An optimal zeitgeber is found when C (40) is less than a threshold cost C_{T} (41).

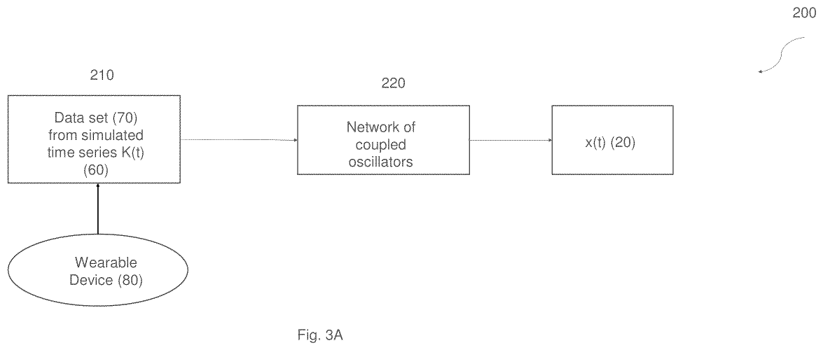

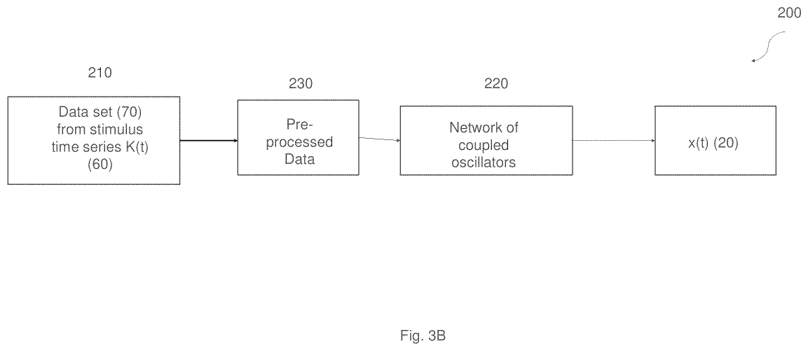

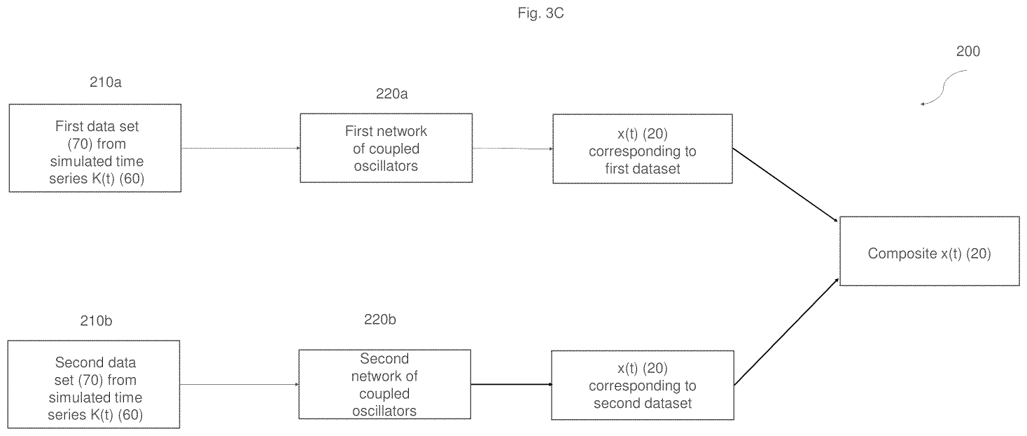

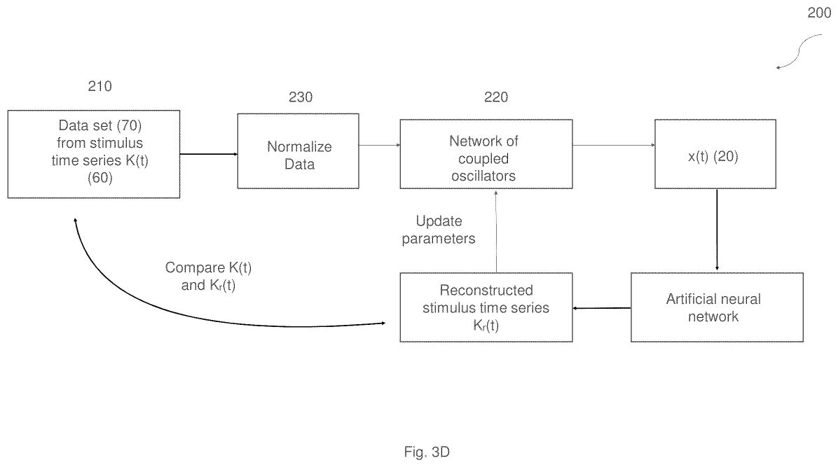

Referring to FIG. 3A, in an embodiment, a method to simulate x(t) (20) in response to a prescribed stimulus history K(t) (60), either at time t or over a time range [tᵢ, tⱼ] (200) is comprised of the steps of receiving at least one data set (70) related to at least one stimulus history K(t) (60) (210); providing the data set (70) to a network of nonlinear coupled oscillators, represented by nodes connected by edges, over a period of time (220). Referring to FIG. 3B, in one embodiment, the data set (70) is pre-processed (230) before it is provided to the network of coupled oscillators. In one embodiment, pre-processed means normalize.

In one embodiment, the network of coupled oscillators is modeled as modified phase oscillators. In another embodiment, the network of coupled oscillators is modeled as Kuramoto oscillators. Referring to FIG. 3A, in one embodiment, the stimulus time series is a data set (70) provided by a wearable device (80) that collects data on the user's heart rate, activity, light exposure, sleep cycle, amongst others. In one embodiment, the stimulus time series is a prescribed pattern of zeitgebers that has been selected to phase shift the clock, and not drawn from wearables.

The network of coupled oscillators represents and behaves like neurons in the brain's SCN in a way that parallels how light input is received by the brain (e.g., brighter light is a stronger stimulus which has more action potential firing in a neuron). The coupling of these oscillators allows for the extraction of a “collective phase” and “collective amplitude” which together can comprise a representation of circadian state.

In one embodiment, the data set (70) may be activity or actigraphy data. In one embodiment, the data set (70) may be heart rate data. In one embodiment, the data set may be light data. In one embodiment, the data set may be comprised of data that is actigraphy data, heart rate data and light data. In one embodiment, actigraphy data is normalized between 0 and 1.

In an embodiment, actigraphy data is uncorrelated from heart rate data to account for the correlation between heart rate and activity to create a heart rate data set with the effects of activity removed. The corrected heart rate set is normalized between 0 and 1. In an embodiment, light data is normalized between 0 and 1; when the data set provides data for continuous light, then the light data set is weighted for the earliest light to be most important.

Referring to FIG. 3C, in an embodiment, multiple data sets (70a, 70b, 70c . . . 70x) may be received; where confidence in at least one data set (70a, 70b, 70c . . . 70x) (230) is greater than the confidence in at least one other data set (70a, 70b, 70c . . . 70x). Here, circadian state trajectories (x_{A}(t), x_{B}(t), x_{C}(t) . . . ) determined using data sets (70a, 70b, 70c . . . 70x) (230) having a higher confidence are upweighted, for instance, given x_{A}(t) arrived at using a heart rate stimulus time series and X_{B}(t) arrived at using a temperature stimulus time series, and assuming that there is missing data from hours 20 to 30 for x_{A}(t) but not x_{B}(t), X_{B}(t) would be prioritized during the period of time when x_{A}(t) is missing data. In an embodiment, the filtering method used for upweighting and down-weighting data sets having varying confidences is a Kalman filter.

Referring to FIG. 3D, in one embodiment, the parameters of the network of coupled nonlinear oscillators, such as their coupling strength, are tuned using an autoencoder neural net. In particular, the output x(t) (20) is provided as input to a neural net and used to construct the stimulus history K(t) (60), arriving at an approximation Kᵣ(t). The parameters in the network of coupled nonlinear oscillators are then updated to reduce the difference between K(t) (60) and Kᵣ(t).

In one embodiment, the network of coupled nonlinear oscillators is represented by a smaller set of equations which approximate its behavior, such as a dimensional reduction, or a type of limit cycle oscillator, such as a van der Pol oscillator.

In one embodiment, cost (40) may be a phase cost, Cₚₕₐₛₑ (40a). A phase cost is determined by the difference between the simulated trajectory x(t) (20) and the target cycle y(t) (30), either at time t or over a time range [tᵢ, tⱼ].

In an embodiment, cost (40) may be a feasibility cost, C_{feasibility} (40b). A feasibility cost (40b) is determined by the zeitgeber K(t) (60), either at time t or over a time range [tᵢ, tⱼ], and is a weighted sum of occurrences when bright light is recommended when the sunlight is unavailable; and when darkness is recommended during sunlight hours.

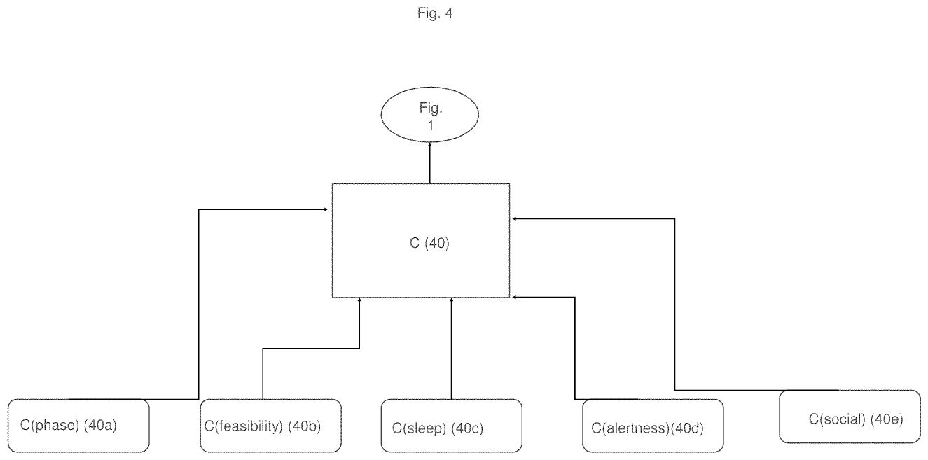

Referring to FIG. 4, in an embodiment, cost (40) may be sleep duration cost, Cₛₗₑₑₚ, (40c). Sleep duration cost (40c) is determined by a simulated sleep model output W(t), either at time t or over a time range [tᵢ, tⱼ]. W(t) is the sleep/wake status of an individual over the course of a trajectory which is determined using the zeitgeber schedule K(t) (60) and the circadian state x(t) (20) as inputs into a sleep model such as the two-process model of sleep provided below.

W(t) is a simulated pattern of sleep and wake as a function of time. W(t)=0 if an individual is predicted, by a sleep model, to be asleep at time tₖ. W(tₖ)=1 if an individual is predicted, by a sleep model, to be awake at time tₖ. W(t) is a function of x(t) (20).

In an embodiment, cost (40) may be alertness cost, Cₐₗₑᵣₜₙₑₛₛ, (40d). Alertness cost (40d) is determined by the simulated alertness, A(t), either at time t or over a time range [tᵢ, tⱼ]. This simulated alertness is determined using the zeitgeber schedule K(t) (60) and the circadian state x(t) (20) as inputs into a fatigue model. This cost reflects the number of hours an individual spends below an alertness threshold over the course of the day. Alertness is calculated using a sleep model that can include caffeine. Certain hours (e.g., working hours) can be weighted as part of this cost.

A(t) is a simulated prediction of alertness as a function of time. A(tₖ) is high if the person is predicted by a fatigue model to be alert at time tₖ. A(tₖ) is low if the person is predicted by a sleep model to be highly fatigued at time tₖ. A(t) is a function of x(t) (20). If F is an embodiment of the fatigue model, A(t)=F(x(t), K(t)).

In one embodiment, cost (40) may be social jetlag cost, C_{social}, (40e). Social jetlag cost (40e) is determined using the simulated sleep model output W(t). This cost reflects the difference between wake-up time and a desired wake-up on a prescribed light schedule such as the wake time predicted to correspond to the target limit cycle y(t) (30).

In one embodiment, cost (40) is at least one taken from the list of: phase cost (40a), feasibility cost (40b), sleep duration (40c), alertness (40d), social jetlag cost (40e).

In an embodiment, a K(t) (60) is a pulse of an activity (e.g., complete 30 minutes of exercise, consume some melatonin, amongst others), where pulse is defined to be a prescription for an activity of any length over a time interval. In an embodiment, a zeitgeber is a period of light exposure and/or lack of light exposure. Light exposure may be light of varying brightness. For example, a K(t) (60) may be a prescription of low light for four hours and darkness for ten. In an embodiment, light having a defined quality is prescribed over a period defined not to exceed an upper limit duration or drop below a lower duration; this is called a light budget. For example, a zeitgeber may be a prescription of light, designed to shift the circadian clock while not exceeding ten hours of continuous darkness at any point. In an embodiment, a zeitgeber may include a pulse of activity and a schedule of light exposure with no budgets. In an embodiment, a zeitgeber may include a pulse of activity and a schedule of light exposure with budgets imposed. In an embodiment, K(t) (60) may be data collected from a wearable device (80).

In an embodiment, the target limit state y(t) (30), when a time zone has been crossed, may be the individual's ideal wake time in the target limit state y(t) (30) where the cost (40) is the number of hours being crossed in the time zone(s). In an embodiment, the y(t) (30), where no time zone is crossed, may be defined by a user's ideal wake time; where the ideal wake time corresponds to the time of day when awakening is predicted to occur when the y(t) (30) is coupled to a sleep model. In an embodiment, the y(t) (30), where an individual moves to work on a night shift, may be a limit state where peak fatigue (or core body temperature minimum) occurs outside of working hours. In an embodiment, the y(t) (30), where an individual has an erratic lighting schedule (e.g., one who works in a different time zone from where they live), may be at a time that peak alertness happens during the night, while allowing sleep during the day.

In one embodiment, a global cost (40) is calculated after having selected a zeitgeber K₀ (50) and integrated x(t) (20) over a time range [tᵢ, tⱼ]; where the global cost (40) is defined as:

C=XC_{A}+YC_{B} . . . +ZC_{N}

where, C_{A}, C_{B} . . . C_{N} each represent different possible criteria of interest, calculated from the circadian state trajectory x(t) (20), the zeitgeber K(t) (60), and any derivative quantities, such as W(t) and A(t), while X, Y, and Z are non-negative weighting constants. In one embodiment, some subset of C_{A}, C_{B} . . . C_{N} may be set to a constant value, such as “1”. It should be noted here that the constant value; further, each C_{A}, C_{B} . . . C_{N} may not be equal in value.

In an embodiment, C_{A}, C_{B} . . . C_{N} is considered according to a defined hierarchy in evaluating a zeitgeber; where each C_{A}, C_{B} . . . C_{N} has a convergence threshold value, and if any cost is above that convergence threshold, all others lower in the hierarchy are set to a maximum constant value. Assuming, that C_{A} is the higher than C_{N} in hierarchy, the following example is provided:

C_{A} is equal to a phase cost, Cₚₕₐₛₑ, (40a); that is, it captures the distance between the circadian state trajectory x(t) (20) and the target limit cycle y(t) (30). If the x(t) (20) is approximately equal to the y(t) (30), then the zeitgeber Kt (60) has been found. However, if x(t) (20) does not approximately equal the y(t) (30), a C_{Ai} is calculated until the cost between x(t) (20) and the y(t) (30) falls below the defined threshold for C_{A}. Once this threshold criterion is met for C_{A}, each subsequent, C_{N}, in hierarchy turn, is then allowed to decrease as new zeitgebers K(t) (60) are sampled until all threshold criteria are met, or some other convergence criterion, such as maximum number of iterations, is reached. In an embodiment, cost is at least one taken from the list consisting of phase cost (40a), feasibility cost (40b), sleep duration cost (40c), alertness cost (40d), social jet lag cost (40e).

In an embodiment, the hierarchy of cost (40), in the case where a time zone is crossed is: phase cost (40a), feasibility cost (40b), sleep duration cost (40c), alertness cost (40d). In an embodiment, the hierarchy of cost (40), in the case of shift work is: phase cost (40a), sleep duration cost (40c), alertness cost (during shift hours) (40d), feasibility cost (40b). In an embodiment, the hierarchy of cost (40), in the case where sleep is shifting to a preferred time is: phase cost (40a), feasibility cost (40b), sleep duration cost (40c), social jet lag cost (40e), alertness cost (40d).

In another embodiment, a global cost (40) is calculated after having selected a zeitgeber K₀ (50) and integrated x(t) (20) over a time range [tᵢ, tⱼ]; where the global cost (40) is defined as:

C=XC_{A}+YC_{B} . . . +ZC_{N}

where, C_{A}, C_{B} . . . C_{N} each represent different possible criteria of interest, calculated from the circadian state trajectory x(t) (20), the zeitgeber K(t) (60), and any derivative quantities, such as W(t) and A(t), while X, Y, and Z are non-negative weighting constants. However, no hierarchy of costs are applied, and the global cost simply reflects the raw sum of the quantities of interest. In one embodiment, cost (40) is at least one taken from the list of: phase cost (40a), feasibility cost (40b), sleep duration (40c), alertness (40d), social jetlag cost (40e).

In another embodiment, a global or total cost (40) is used to evaluate a zeitgeber, at time t, as:

C=C_{A}×C_{B}× . . . C_{N}

In an embodiment, C_{A}, C_{B}, . . . C_{N} is considered in prescribing a zeitgeber; where each C_{A}, C_{B}, . . . C_{N} has a minimum and maximum value. In one embodiment, cost (40) is at least one taken from the list of: phase cost (40a), feasibility cost (40b), sleep duration (40c), alertness (40d), social jetlag cost (40e).

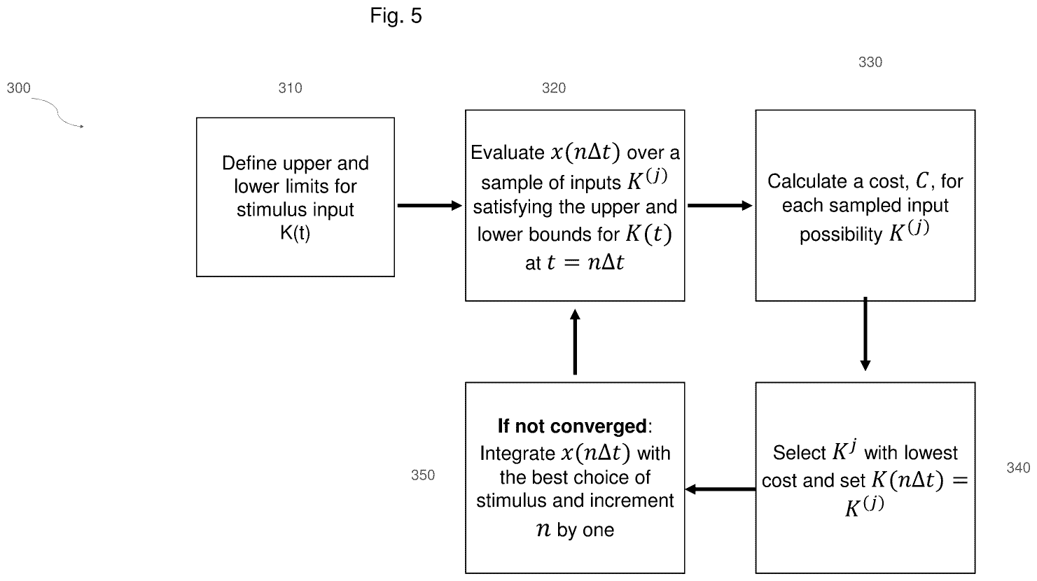

Referring to FIG. 5, in an embodiment, a method to determine a K(t) (300) comprised of the steps of: define a vector defining a zeitgeber event at [t, tᵢ]; define K(t); providing lower and upper bounds for K(t) (310); using a sampling method, having the components of K(t), sample within the lower and upper bounds prescribed for K(t) (320); calculating the cost from the circadian state trajectory and the zeitgeber, C=(x(t), K(t)) (330); interpolating sample points to identify possible locations of minima for C (340); resampling K(t) in identified possible minima locations at a denser resolution and repeating the interpolation step (350). In an embodiment, the vector defining a zeitgeber may include a duration of light, a duration of dark, and/or a timing of a zeitgeber event. In an embodiment, the upper and lower bounds of K(t) (300) are provided as follows: for light and dark duration, the bounds are set to be the minimum and maximum allowable time spent in either dark or light; however, the first and last light and dark duration have no lower bounds; for activities specified once every N hours, such as caffeine and exercise, the lower and upper bounds are set to be k×N and (k+1)×N; where k is the specified activity.

In an embodiment, the method to determine K(t) (300) is repeated until a defined convergence limit is reached or a maximum number of iterations is met. In an embodiment, the sampling method is Latin hypercube sampling. In an embodiment, sample points are interpolated as polynomials to identify rough locations of minima. In an embodiment, sample points are interpolated using more sophisticated curve fitting functions, such as an artificial neural net, to identify rough locations of minima.

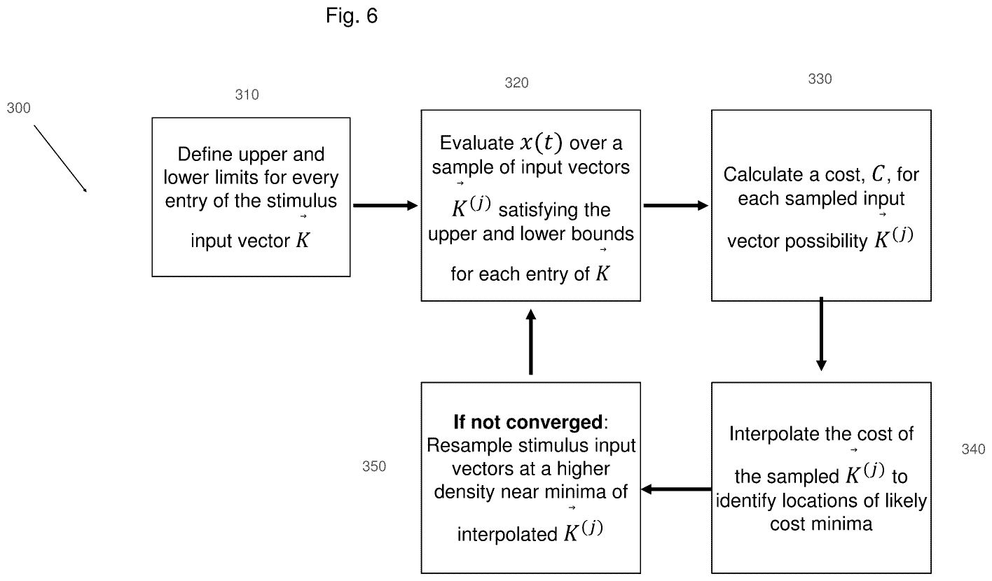

Referring to FIG. 6, in another embodiment, a method to determine a K(t) (300) is comprised of the steps of: calculate the components of x₀(310); sample from a range of possible zeitgebers K⁽¹⁾, K⁽²⁾, . . . K^{(N)} for a single time step, where each K corresponds to a different zeitgeber presentation, such as light of a certain quality, or the presence/absence of an activity (320); calculate the circadian state trajectory x(t) for that time step for all K⁽ⁱ⁾; calculate the cost C⁽ⁱ⁾ of each K⁽ⁱ⁾ and choose the K⁽ⁱ⁾ with the lowest cost (330); set K(t₀) to be the chosen zeitgeber values with lowest cost (340); repeat the process for all remaining time steps.

As will be appreciated by one skilled in the art, aspects of the present invention may be embodied as a system, method or computer product. Accordingly, aspects of the present invention may take the form of an entirely hardware embodiment, entirely software embodiment (including firmware, resident software, micro-code, etc.) or an embodiment combining software and hardware aspects. Further aspects of this invention may take the form of a computer program embodied in one or more readable medium having computer readable program code/instructions thereon. Program code embodied on computer-readable medium may be transmitted using any appropriate medium, including but not limited to wireless, wireline, optical fiber cable, RF, etc., or any suitable combination of the foregoing. The computer code may be executed entirely on a user's computer, partly on the user's computer, as a standalone software package, a cloud service, partly on the user's computer and partly on a remote computer or entirely on a remote computer, remote or cloud-based server.

We claim as our invention:

1. A method to minimize cost to approximately entrain to a target circadian state is comprised of the steps:
(a) identifying an initial circadian state, x₀;
(b) identifying a target circadian state trajectory, y(t);
(c) providing an initial zeitgeber, K0(t), either at a time or over a time range;
(d) a method to simulate a circadian state trajectory, x(t), in response to a zeitgeber, either at a time or over a time range is comprised of (i) receiving at least one data set; (ii) a method (b) to develop at least one zeigeber history, K(t) using the data set; providing the data set to a network of coupled oscillators, represented as nodes connected by edges, over a time period [ti, tj];
(e) a method to determine a cost C(x(t), y(t), K(t)), either over the same time or time range in (d), for x(t); where C is the cost derived from x(t), y(t) and K(t);
(f) where the method to determine K(t), either over the same time or over a time range is comprised of: (i) providing lower and upper bounds for K(t); (ii) sampling components of K(t) within the lower and upper bounds; (iii) calculating the cost, C, from the circadian state trajectory, the target trajectory, and the zeitgeber, C(x(t), y(t), K(t)); (iv) interpolating the cost values at the sampled K(t) to identify possible locations of minima for C; (v) determining a new K(t) in identified minima locations at a denser resolution;
(g) updating K(t) in response to C, repeating steps (d) and (e) until a convergence criteria is met.

2. The method according to claim 1, where K(t) is comprised of a duration of light, a duration of dark, and/or a timing of a zeitgeber event.

3. (canceled)

4. The method according to claim 1, where the method to simulate x(t) in response to a zeitgeber, either at a time or over a time range, is further comprised of the step of pre-processing the data set prior to providing the data set to the network of coupled oscillators.

5. The method of claim to claim 1, where the parameters of the coupled oscillators are tuned using an autoencoder neural network.

6. The method according to claim to claim 1, where the network of coupled oscillators is modeled as a macroscopic reduction of a coupled phase oscillator network.

7. The method according to claim to claim 1, where the data set is a data set that includes at least one taken from: actigraphy data set, heart rate data set, light data set, temperature data set.

8. The method according to claim 7, where the data set is provided by a wearable device.

9. The method according to claim 7, where at least a first x(t) derived from a first data set and a second x(t) is derived from a second data set; where the first x(t) and the second x(t) is weighted by confidence in the quality of the data.

10. The method according to claim 7, where at least a first data set and a second data set are combined using a Kalman filter.

11. (canceled)

12. The method according to claim 1, where the method to provide the lower and upper bounds for K(t) is comprised of the steps:
(a) for light and dark duration, the bounds are set to be the minimum and maximum allowable time spent in either dark or light; however, the first and last light and dark duration have no lower bounds;
(b) for activities specified once every N hours, the lower and upper bounds are set to be k×N and (k+1)×N; where k is the specified activity.

13. The method according to claim 1, where sampling components of K(t) with the upper and lower bound is Latin hypercube sampling.

14. The method according to claim 1, where sampled cost values are interpolated with polynomials, trigonometry functions, or neural networks.

15. The method according to claim 1, where the method to determine a zeitgeber, either at a time or over a time range is comprised of the steps:
(a) calculate the components of x₀;
(b) sample from a range of possible zeitgebers K⁽¹⁾, K⁽²⁾, . . . K^{(N)}, where each zeitgeber choice K⁽ⁱ⁾ represents a different choice of light or other stimulus for a single time step;
(c) simulate x(t) over a single time step to get x₁ for each sampled Kⁱ;
(d) calculate the cost for all choices Kⁱ, choose the Kⁱ with the lowest cost, Cⁱ; set K(t₀) to be the chosen zeitgeber values with lowest cost;
(e) repeat these steps for xₙ.

16. The method according to claim 1, where the cost to approximately entrain to a target circadian state is minimized when C(x(t), y(t), K(t)) is less than a threshold value of cost.

17. The method according to claim 16, where C(x(t), y(t), K(t)) is:
C=XC_{A}+YC_{B} . . . +ZC_{N}
where, C_{A}, C_{B} . . . C_{N} each represent different possible criteria of interest and X, Y, Z represent weightings of these costs.

18. The method according to claim 17, where C_{A}, C_{B} . . . C_{N} is considered according to a defined hierarchy in evaluating a zeitgeber.

19. The method according to claim 17, where C_{N} is at least one taken from the list of: phase cost, feasibility cost, sleep duration, alertness, social jetlag cost.

20. The method according to claim 16, where C(x(t), y(t), K(t)) is:
C=C_{A}×C_{B}× . . . C_{N}
where each C_{A}, C_{B}, . . . C_{N} has a minimum and maximum value.

21. The method according to claim 20, where C_{N} is at least one taken from the list of: phase cost, feasibility cost, sleep duration, alertness, social jetlag cost.
